(19) United States
(12) Patent Application Publication
(10) Pub. No.: US 2019/0135879 A1
(43) Pub. Date: May 9, 2019

(54) GENE REGULATING BIN2 FUNCTION AND TRANSGENIC PLANT TRANSFORMED BY THE GENE

(71) Applicant: SEOUL NATIONAL UNIVERSITY R&DB FOUNDATION, Seoul (KR)

(72) Inventors: Sunghwa Choe, Gyeonggi-Do (KR); Yu Jeong Jeong, Seoul (KR)

(21) Appl. No.: 16/204,754

(22) Filed: Nov. 29, 2018

Related U.S. Application Data
(63) Continuation of application No. 15/470,269, filed on Mar. 27, 2017, now abandoned.

(30) Foreign Application Priority Data
Mar. 25, 2016 (KR) 10-2016-0036392

(51) Int. Cl. C07K 14/415; C12N 15/82

(52) U.S. Cl. CPC C07K 14/415; C12N 15/8298; C12N 15/8261

(57) ABSTRACT

The present invention relates to a gene that regulates BIN2 function and a transgenic plant into which the gene is introduced. When the gene of the present invention is introduced into an economically useful crop, it is advantageous to produce a high value-added plant with excellent productivity.

### CROSS REFERENCE TO RELATED APPLICATIONS

This application is a continuation application of U.S. application Ser. No. 15/470,269, filed on 27 Mar. 2017, which claims the benefit and priority to Korean Patent Application No. 10-2016-0036392, filed on 25 Mar. 2016. The entire disclosures of the applications identified in this paragraph are incorporated herein by references.

### TECHNICAL FIELD

The present invention relates to a novel gene that regulates BIN2, a major negative factor of brassinosteroids (BRs), and a transgenic plant into which the gene is introduced.

### BACKGROUND

Glycogen Synthase Kinase 3 (GSK3)-like kinases regulate a broad range of fundamental biological processes in both humans and plants. In mammal, GSK3 plays roles in multiple cellular processes and signaling pathways that are involved in cell proliferation, differentiation, development, several human diseases including Alzheimer's diseases, several signaling pathways including Wnt, insulin, notch, hedgehog signaling, mitosis, and apoptosis (Doble and woodgett, 2003; Kockeritz et al., 2006; Jin et al., 2009; Kim et al., 2009b; Wu and Pan, 2010).

In resting cells, GSK3 is a constitutively active kinase that phosphorylates a wide array of protein substrates to directly inhibit their biochemical activities, to interfere with their subcellular localization, or to promote their degradation (Ali et al., 2001). In plants, GSK3 kinases play roles in cell expansion, floral organ development, stomata development, light response, as well as in responses to biotic and abiotic stresses. GSK3 may mediate the crosstalk between BR signaling and other hormone signaling pathways (Vert et al., 2008; Zhang et al., 2009).

BR-INSENSITIVE 2 (BIN2) was the first plant GSK3-like kinase characterized from genetic screening and plays a negative role in the signal transduction pathway of brassinosteroids (BRs) (Clouse and Sasse, 1998; Li et al., 2001; Li and Nam, 2002). Genetic and biochemical studies suggested that BIN2 is a constitutively active kinase that phosphorylates BR responsive transcription factors BRI1 EMS suppressor 1 (BES1/BZR2) and BRASSINAZOLE RESISTANT 1 (BZR1) to affect their nuclear localization (Gampala et al., 2007; He et al., 2005; Ryu et al., 2007; Yin et al., 2005), to inhibit DNA binding (Vert and Chory, 2006), and to target BZR1 for protein degradation (He et al., 2002; Wang et al., 2002; Yin et al., 2002; Zhao et al., 2002), thus blocking the BR signal transduction into the nucleus. Recent studies suggested that BIN2 kinase activity and protein level are negatively regulated by BR signaling through respectively dephosphorylation of a conserved tyrosine residue, proteasome-mediated protein degradation mechanism (Kim et al., 2009a; Peng et al., 2008) and different subcellular localization of BIN2 might be important mechanisms to regulate its activity (Peng et al., 2008; Vert and Chory, 2006).

Other mechanisms, such as proteasome-mediated protein degradation and differential subcellular distribution, might also be involved in regulating the activity of the animal GSK3 kinase (Bijur and Jope, 2001; Diehl et al., 1998; Failor et al., 2007, Meares and Jope, 2007). Protein degradation via ubiquitination is an important post-translational regulatory mechanism in eukaryotes (Dreher et al., 2007; Moon and Callis., 2004; Vierstra, 2009). In plant, the ubiquitin/26S proteasome pathway is involved in mediation of various hormone signals (Dharmasiri et al., 2005; Kepinski and Leyser, 2005; Katsir et al., 2008; Santner and Estelle, 2009; Schwechheimer and Willige, 2009).

The Arabidopsis genome encodes more than 1,400 different E3 ligases, including more than 700 F-box proteins. They reported that F-box proteins have been identified in plants which are involving in hormone signal transduction and biological processes (Ruegger et al., 1998; Xie et al., 1998; Vierstra, 2003). They are yet to be discovered how the activity of BIN2 is regulated in plants. It was reported that BR treatment induces proteasome-mediated degradation of BIN2, but still is necessary to identify the participating E3 ligases that may ubiquitinated BIN2.

The inventors of the present invention discovered an E3 ligase having an F-box motif that specifically binds to and cleaves BIN2 protein, thereby completing the present invention.

### SUMMARY

The present invention aims to provide a base sequence of an F-box gene that regulates BIN2 function and an amino acid sequence thereof, and to provide a transgenic plant into which the gene is introduced.

In order to accomplish the above object, the present invention provides a gene comprising the nucleotide sequence of SEQ ID NO: 1.

The present invention also provides a protein comprising the amino acid sequence of SEQ ID NO: 2.

The present invention also provides a gene comprising the nucleotide sequence of SEQ ID NO: 3.

The present invention also provides a protein comprising the amino acid sequence of SEQ ID NO: 4.

The present invention also provides a gene encoding the protein.

The present invention also provides a transgenic plant into which the gene is introduced.

Since the F-box protein of the present invention accelerates the development of plant growth by decomposing BIN2, introduction of the F-box gene into an economically useful crop can produce a high value-added plant with excellent productivity.

### BRIEF DESCRIPTION OF THE DRAWINGS

FIGS. 1A, 1B, 1C, 1D and 1E relate to the regulation of Arabidopsis BIN2 through proteasome-mediated protein degradation;

FIG. 1A is schematic representations of the vector constructs of 35S:BIN2-HA 35S:bin2-6D-HA and 35S:BIN2KD-HA transgenic plants. The illustrated vector features include: LB, T-DNA left border; RB, T-DNA right border; 35S, CaMV 35S promoter; YFP, yellow fluorescent protein; CFP, HA; hemagglutinin epitope tags;

FIG. 1B is phenotypes of BIN2, bin2-6D (BIN2^{E264K}), and BIN2KD (BIN2^{K69R}) transgenic plants grown in soil for 3 weeks under long day conditions. BIN2-HA transgenic lines displayed dwarf phenotypes similar to the bin2-6D-HA line. Scale bar, 1 cm;

FIG. 1C shows phenotypic changes of the BIN2 mutant after BL treatment. A BIN2-HA transgenic line responds to BL treatment whereas a dwarfed bin2-6D-HA line is BL-insensitive. From left to right are bin2-6D:HA (upper) and BIN2:HA (lower) transgenic seedlings grown on 1 μM BL-containing medium for 0, 2, 4, and 8 d;

FIG. 1D shows the amount of expressed protein of BIN2 in terms of time and concentration upon BL treatment. The dosage-dependency of the BL-induced BIN2 decrease;

FIG. 1E shows that BIN2 overexpressed plants treated with BL exhibit a reduced amount of BIN2 protein and an E3 ligase involved in ubiquitin/proteasome mediated degradation specific to BIN2 through MG132 treatment. BIN2-HA seedlings were grown on medium with 1 μM BRZ for 2 weeks and transferred to liquid medium containing 1 μM BL and/or 10 μM MG132 for 30 min. The amount of BIN2-HA protein in various treated seedlings was analyzed by the IP/Western analysis and the relative amount of total proteins was estimated by Ponceau S staining.

FIGS. 2A, 2B, 2C, 2D and 2E relate to identification and characterization of F-box protein using immunoprecipitation (IP) coupled with MALDI-TOF mass spectrometry;

FIGS. 2A and 2B show total protein extracts from FIG. 1E condition without MG132, immunoprecipitated with high-affinity immobilized HA-antibody and then analyzed by silver staining/Western;

FIG. 2C is a table showing unique peptides and sequence coverage of the BRF1 and BRF2 protein identified by MALDI-TOF MS;

FIG. 2D shows the amino acid sequences of the identified Arabidopsis BRF1 and BRF2 proteins (SEQ ID NOs: 2 and 4). The number of peptides determined by MS/MS analysis is shown in red;

FIG. 2E shows the interaction between BIN2 and F-box by the yeast two-hybrid system. It is a yeast two-hybrid assays showing the interaction of BIN2 and BRF1. Two clones of yeast containing each combination of GBK (BD) and GAD (AD) vectors were grown on medium with or without His. pGBKT7, pGADT7 empty vector was used as a negative control.

FIGS. 3A, 3B and 3C relate to the identification of the BIN2 protein determined by LC-MS/MS analysis;

FIG. 3A shows tandem mass spectrum obtained from a doubly charged ion with monoisotopic m/z=660.3462⁺² (corresponding to LLQYSPSLR(SEQ ID NO: 5)), m/z=617.3⁺² (corresponding to MPPEAIDFASR(SEQ ID NO: 6)), m/z=679.3⁺² (corresponding to QEVAGSSPELVNK(SEQ ID NO: 7)), m/z=617.3⁺² (corresponding to VLGTPTREEIR(SEQ ID NO: 8)), m/z=434.9⁺² (corresponding to VLKHYSSANQR(SEQ ID NO: 9)), and m/z=705.4⁺² (corresponding to VVGTGSFGIVFQAK(SEQ ID NO: 10)) present in band 1 protein of FIG. 2A;

FIG. 3B shows peptide ID by LC-MS/MS analysis. * m/z: mass to charge ratio; MC #: trypsin miss cleavaged; RT: retention time on reverse phase C18 column; z: multiple charge number. Peptides 1-6 of correspond to SEQ ID NOs: 5-10, respectively;

FIG. 3C shows the BIN2 proteins, amino acid sequence coverage and the number of determined peptides identified by MS/MS analysis in red (SEQ ID NO: 11).

FIGS. 4A and 4B shows a list of the BRF1 and BRF2 signature peptides derived from BL-induced BIN2 protein in immunoprecipitation coupled with MALDI-TOF MS (SEQ ID NOs: 12-21 and 22-41).

FIGS. 5A and 5B to phylogeny and sequence alignment of the Arabidopsis BRF1 and BRF2 homologene;

FIG. 5A shows an amino acid sequence based phylogeny of homologene including BRF1 and BRF2 from Arabidopsis. thaliana. Designations on the left identify the group and the accession number for each protein. The bar represents the branch length equivalent to 3.0 amino acid changes per residue;

FIG. 5B shows the amino acid sequence alignment of BRF1 and BRF2 from Arabidopsis thaliana. The leucine-rich repeats (LRRs) in the central region, F-box motif in the N-terminal region, F-box and leucine rich repeat (FBD) in the C-terminal region are indicated. Conserved and similar amino acids are shown in black and gray boxes, respectively. The GenBank accession numbers for Arabidopsis BRF1 and BRF2 homologs are as follows: BRF1 (gi|15242584; NP_201102.1) (SEQ ID NO: 2), BRF2 (gi|15241209; NP_200480.1) (SEQ ID NO: 4), AT5G56410 (gi|15239385; NP_200452.1) (SEQ ID NO: 42), AT2G26860 (gi|42570929; NP_973538.1) (SEQ ID NO: 43), AT5G60610 (gi|15239385; NP_200869.1) (SEQ ID NO: 44), AT5G56700 (gi|15241898; NP_200481.1) (SEQ ID NO: 45).

### DETAILED DESCRIPTION

Hereinafter, embodiments of the present invention will be described in detail. These embodiments are only for explaining the present invention in more detail.

A. Materials and Methods

1. Plant Materials and Growth Conditions

Arabidopsis thaliana ecotype Columbia-0 (Col-0) was used as wild-type controls and genetic backgrounds of transgenic lines, BIN2-HA, bin2-6D (BIN2^{E264K})-HA, BIN2KD (BIN2^{K69R})-HA. All fusion proteins were expressed by the 35S promoter.

Arabidopsis seeds were sterilized and germinated on agar-solidified 1/2 Murashige and Skoog (MS, Duchefa #M0222) with 1% sucrose. After stratification at 4° C. for 3 days, the wild type and mutant plants were grown under a photoperiod of a long day condition (16 h light/8 h dark) at 23° C.

2. Plasmid Constructs and Transgenic Plants

The full length cDNA fragments of BIN2, bin2-6D (BIN2_{E264K}), and BIN2KD (BIN2^{K69R}) without stop codon were amplified by PCR using gene specific primers, BIN2-F and BIN2-R (BIN2-F; 5′-CACCATGGCTGATGATAAGGAGATGC-3′ and BIN2-R; 5′-AGTTCCAGATTCAAGAAGCT-3′) (SEQ ID NOs: 46-47).

PCR products were subcloned into pENTR/SD/D-TOPO (Invitrogen) using LR Clonase (Life Technologies) into the binary vector pEarley Gate 101. The 35S promoter was used for constant expression in this plant transformation vector, and YFP-HA (yellow fluorescent protein), a reporter protein, and hemagglutinin were fused to confirm expression at the C-terminus. All constructs were introduced into Arabidopsis by Agrobacterium strain GV3101.

The floral dip method (Clough and Bent, 1998) was used to introduce the transgene into wild-type Col-0 plants generating, bin2-6D-HA, BIN2-HA and BIN2KD-HA, respectively. Homozygous transgenic plants were selected from the T3 generation based on resistance to 50 mg/mL Basta (DL-Phosphinothricin, Duchefa Biochemie) and expression of the transgene was confirmed by Western blot.

3. Immunoprecipitation of HA-Tagged Protein

Seedlings from 1-week-old plants were harvested and ground to powder in N₂ and mixed with the extraction buffer containing 50 mM Tris-Cl (pH 7.5), 100 mM NaCl, 10 mM MgCl₂, 1 mM EDTA, 10% glycerol 0.1% NP-40, 1 mM phenymethylsulfonyl fluoride (PMSF), and 1× protease inhibitor cocktail (GenDEPOT) 20 min at 4° C.

The IP experiments were performed using HA Tag IP/Co-IP Kit according to the manufacturer's protocol (Thermo Pierce).

The supernatant was incubated with prewashed anti-HA agarose beads for 4 h at 4° C. with gentle shaking. The beads were collected and washed three times with TBS plus 0.05% Tween-20 detergent (TBST), and dissolved in 2×SDS sample buffer. The proteins were separated by 10% SDS-PAGE, and Anti-HA (1:2,000, AbCam #9110) antibody was used to detect BIN2-HA.

4. BL Treatment Assay

Seeds of various transgenic lines were germinated on Murashige and Skoog medium (1/2 MS) with 2 μM BRZ (Sigma), a BR biosynthesis inhibitor, and grown under a long day condition at 23° C. for 1 week. The seedlings were transferred to liquid 1/2 MS medium with 1% sucrose containing various concentrations of BL (24-epibrassinolide, Sigma #E1641), and incubated for the indicated times before being removed for Western analysis.

To investigate the BL-Induced BIN2 degradation, BRZ (Sekimata et al., 2001) treated seedlings were incubated in liquid 1/2 MS medium containing 1 μM BL and/or 20 μM MG132 (Sigma), a specific 26S proteasome inhibitor (Rock et al., 1994) for 30 min.

5. Identification of Protein Interacting with BIN2 by MALDI-TOP MS

Seeds of transgenic lines, BIN2-HA, bin2-6D-HA, and BIN2KD-HA, were germinated on agar-solidified 1/2 MS with 1% sucrose supplemented with 1 μM BRZ and grown under a long day condition at 23° C. for 2 week. The seedlings were transferred them to liquid medium (1/2 MS with 1% sucrose) with 1 μM BL, and incubated for 30 min.

These seedlings were ground to powder in liquid nitrogen and solubilized with protein extraction buffer containing 50 mM Tris-Cl (pH 7.5), 100 mM NaCl, 10 mM MgCl₂, 1 mM EDTA, 10% glycerol 0.1% NP-40, 1 mM PMSF, and 1× Protease inhibitor cocktail (GenDEPOT). The extracts were centrifuged at 20,000×g for 20 min, and the resulting supernatants were collected and incubated with pretreated anti-HA agarose bead (Thermo Pierce) at 4° C. After incubation, the agarose beads were collected by centrifugation at 800×g for 30 sec and washed three times with extraction buffer.

Immunoprecipitation (IP) complex was eluted by elution buffer containing 50 mM Tris-HCl (pH 7.5), 50 mM DTT, 1 mM EDTA, 10% glycerol, 1% SDS, and 0.01% bromophenol blue, and then separated in a 10% SDS-PAGE gel, and immunoblot analysis. Antibodies against HA (Abcam) were used for protein detection. For visualization of protein bands, the gel was stained with the SilverQuest Silver Staining Kit (Invitrogen) according to the manufacturers protocol.

The gel lane with separated bin2-6D-HA proteins was cut into a single slices, and then in-gel tryptic digestion and peptide desalting, the extracted peptides were analyzed by MALDI-TOF mass spectrometry on an Ultraflex III TOF/TOF (Bruker Daltonics, Germany).

The search for identity proteins was performed using the search engine MASCOT protein database (www.matrixscience.com) by scanning the current version of NCBI sequence database. Proteins identified in the bin2-6D-HA samples are considered real BIN2 interacting proteins.

6. Identification of BIN2 using Liquid Chromatography/LTQ-Orbitrap Mass Spectrometer (LC/LTQ-Orbitrap MS)

After IP in bin2-6D-HA transgenic seedling, the eluted proteins were separated in a 10% SDS-PAGE.

After gel staining using SilverQuest Silver Staining Kit, BIN2 protein bands subjected to in-gel tryptic digestion, and the peptides were desalted and then analyzed by LC-MS/MS by using LTQ Orbitrap Velos mass spectrometer (Thermo Scientific) using the same method as described by Zhao at al., 2011. The LTQ Orbitrap Velos was operated in a CID top 10 mode essentially as described (Olsen et al., 2009).

Acquired data files containing MS/MS spectra were searched against the UniProt database using the software MaxQuant with a false discovery rate (FDR)<1%. After peptide identification, uniprot IDs were converted into Arabidopsis accession numbers using the uniprot website (www.uniprot.org).

7. Yeast Two-Hybrid Assay of the BIN2-BRF1 Interaction

Yeast two-hybrid assay was conducted following the Matchmaker Gold Yeast Two-Hybrid System manufacturer's protocol (Clontech) for testing the BIN2 and BRF1 interaction.

To make the yeast two-hybrid assay constructs, the full-length cDNA fragments of BIN2 and BRF1 were PCR amplified with specific primers, BIN2-F and BIN2-R, BRF1-NdeI-F and BRF1-EcoRI-R, respectively. The PCR products BIN2 and BRF1 were ligated to the prey pGADT7 vector (AD vector) and the bait pGBKT7 vector (DBD vector), respectively. These prey and bait vectors were transformed into the yeast strain AH109, and yeast was grown on SD/-Trp/-Leu medium for 3 days. Transformants were selected on SD/Leu-/Trp- and SD/Leu-/Trp-/His-/1.5 mM 3-amino-1,2,4-triazole (3-AT, Sigma) medium. Empty vectors were used as the negative control.

Yeast two-hybrid analysis performed using primers BIN2-NdeI-F (5′-TCATATGATGGCTCATGATAAGGAGATGCCT-3′), BIN2-EcoRI-R (5′-TGGAATTTCTTAAGTTCCAGATTGATTCAAGAA-3′), BRF1-NdeI-F (5′-CTGCATATGATGGACAAGATCAGTGGGTTTTCT-3′), BRF1-EcoRI-R (5′-CGGGAATTCTCAATAGAATACGCGTTTGCATGT-3′), (NdeI and EcoRI sites are underlined) (SEQ ID NOs: 48-51)

8. Phylogeny Analysis of Arabidopsis BRF1 and BRF2 Homologs

A. thaliana BRF1 (AT5G62970) and BRF2 (AT5G56690) homologenes were identified by searching the NCBI HomoloGene database (www.ncbi.nlm.nih.gov/HomoloGene/) and then selected based on overall similarity.

Homologene uses a pairwise gene comparison approach combined with a guide tree and gene neighborhood conservation to group orthologs (Wheeler et al., 2007). To construct the phylogeny of BRF1, BRF2 and its homologene protein in Arabidopsis, the ClustalW program was used.

B. Result and Discussion

1. The BL-Induced BIN2 Disappearance Involves Proteasome-Mediated Protein Degradation

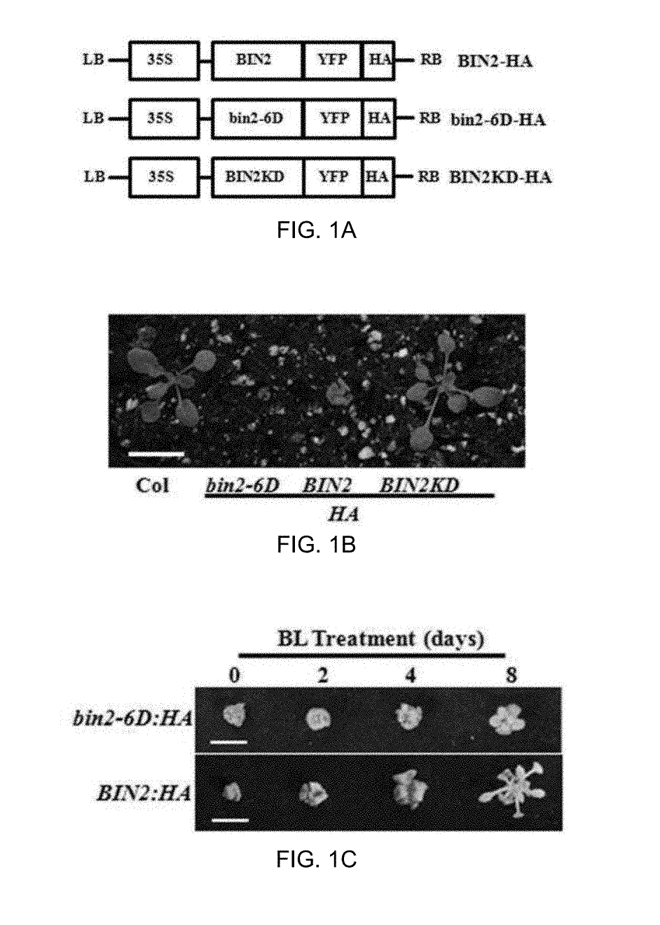

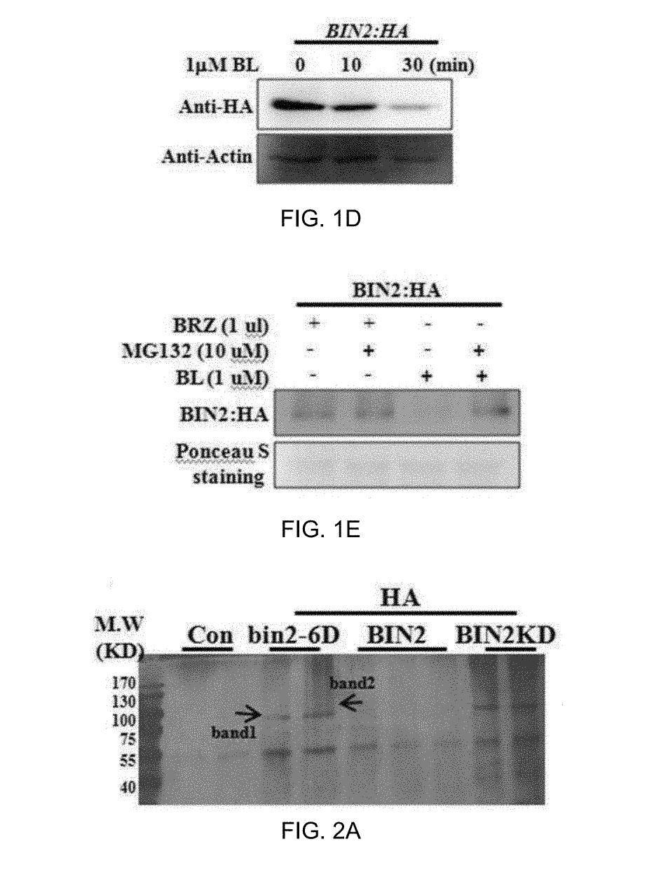

To purify and detect the BIN2 protein and its interacting partner, an E3 ligase regulating the stability of BIN2, we first generated to transgenic Arabidopsis plants overexpressing BIN2-HA, gain-of-function bin2-6D (BIN2^{E264K})-HA, and a kinase-dead loss-of-function BIN2KD (BIN2^{K69R})-HA construct (FIG. 1a).

We have selected transgenic lines of phenotypes for each construct, BIN2-HA, bin2-6D-HA, BIN2KD-HA, examined HA accumulation by Western analyses. C-terminal BIN2-HA, bin2-6D-HA, and BIN2KD-HA fusion proteins were stably expressed in Arabidopsis wild-type plants under the control of the 35S promoter as described above. BIN2-HA and bin2-6D-HA transgenic lines displayed morphologically similar dwarf phenotypes as bin2-1 mutant. To directly test whether BIN2 is regulated at the protein level, we selected a BIN2-HA line that exhibits a weak bin2 phenotype (FIG. 1b). The bin2-6D-HA transgenic lines caused phenotypes typical of BR-insensitive mutants, such as small, dwarf, dark-green and curly leaves and insensitivity to epi-BL like dwf12-1D and ucu1-1/2 and (Choe et al., 2002; Perez-Perez et al., 2002).

To confirm whether the amount of BIN2 is affected by the BRs level, we grew BIN2-HA line and a bin2-6D-HA line on medium containing 2 μM BRZ for 2 weeks and then transferred them to liquid medium with 1 μM epi-BL for 2 to 8 days. The BRZ treatment enhanced a strong bin2 phenotype of the BIN2-HA seedling unlike the bin2-6D-HA seedlings.

The mutation of dwf12-1D and ucu1-1/2 in the TREE motif within the catalytic domain would prevent the phosphorylation of BIN2 by Casein Kinase II (CKII) and its recognition by an E3 ligase, thus making gain of function mutant bin2-1 a more stable protein than its wild-type form (Choe et al., 2002; Perez-Perez et al., 2002).

To test whether the BRZ-stabilized BIN2-HA protein level and phenotype can be affected by BL treatment time, the BRZ-stabilized BIN2-HA protein was rapidly decreased within 10-30 min by BL treatment (FIGS. 1c and 1d). Exogenous application of BRZ increased BIN2 protein, while application of an active BR decreased BIN2 proteins.

Recent studies highlight the importance of the ubiquitin-proteasome system (UPS) action in hormone signaling. UPS-mediated protein degradation has been implicated for plant hormone (Calderbn Villalobos et al., 2012; Fu et al., 2012; Lyzenga et al., 2012; Sheard et al., 2010; Willige et al., 2007). The UPS is also very important for steroid hormone signaling in humans (Lee and Lee, 2012).

A previous report demonstrated that BIN2 kinase of BR signaling is regulated by proteasome-mediated protein degradation (Peng et al., 2008). To confirm whether the BL-induced BIN2 disappearance is caused by proteasome-mediated degradation, we grew the BIN2-HA seedling on 1 μM BRZ-containing medium for 2 weeks and then transferred to liquid medium containing 1 μM BL and/or 20 μM MG132. The seedlings were collected after 30 min incubation for the IP/Western analysis. As shown in FIG. 1e, MG132 treatment effectively blocked the BL-induced BIN2 disappearance and a nullified effect of BL on the BIN2 kinase activity. These results indicate that proteasome-mediated protein degradation constitutes an important regulatory mechanism for restricting the BIN2 activity.

2. BIN2 Interacts with BRF1

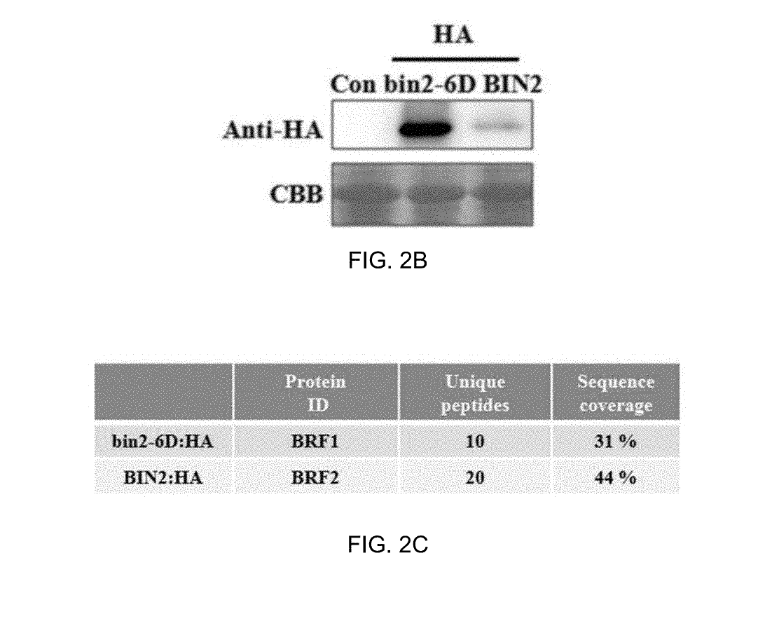

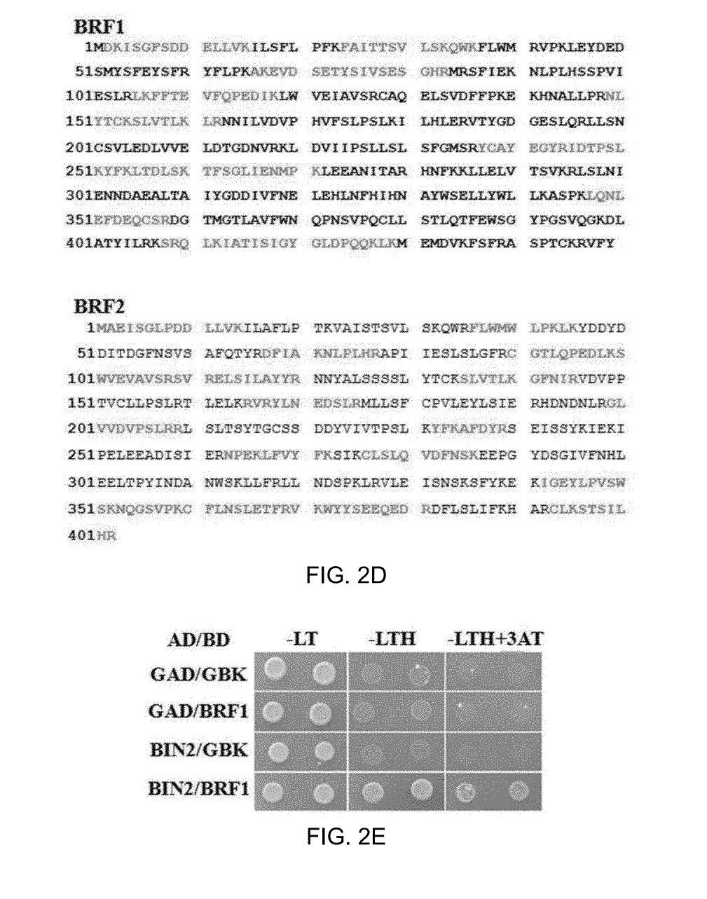

To identify components of an E3 ligase regulating the stability of BIN2, we combined immunoprecipitation of BL-induced BIN2-HA lines with LC/MALDI-TOF/MS-based protein identification. First, we grew transgenic lines, BIN2-HA, bin2-6D-HA, BIN2KD-HA, on medium containing 2 μM BRZ for 2 weeks and then transferred them to liquid medium with 1 μM epi-BL for 30 min. Total soluble proteins extracted from these seedlings were purified using high-affinity columns followed by IP using anti-HA antibodies immobilized anti-HA affinity resin (see Materials and Methods). The purified proteins after IP were separated by SDS-PAGE and analyzed by silver staining (FIG. 2a).

The BIN2 protein was successfully observed in a separate SDS-PAGE gel and recovered with Anti-HA antibody (FIG. 2b). Peptide samples prepared from in-gel digestion of band 1, which appeared to be BIN2 protein, were analyzed by reverse phase liquid chromatography tandem mass spectrometry (LC-MS/MS) using an LTQ-Orbitrap as described “Materials and Methods”.

Tandem mass spectrum obtained from a doubly charged ion with monoisotopic m/z=660.3462⁺² (corresponding to LLQYSPSLR(SEQ ID NO: 5)), m/z=617.3⁺² (corresponding to MPPEAIDFASR(SEQ ID NO: 6)), m/z=679.3⁺² (corresponding to QEVAGSSPELVNK(SEQ ID NO: 7)), m/z=617.3⁺² (corresponding to VLGTPTREEIR(SEQ ID NO: 8)), m/z=434.9⁺² (corresponding to VLKHYSSANQR(SEQ ID NO: 9)), and m/z=705.4⁺² (corresponding to VVGTGSFGIVFQAK(SEQ ID NO: 10)) present in band 1 protein of FIG. 2a.

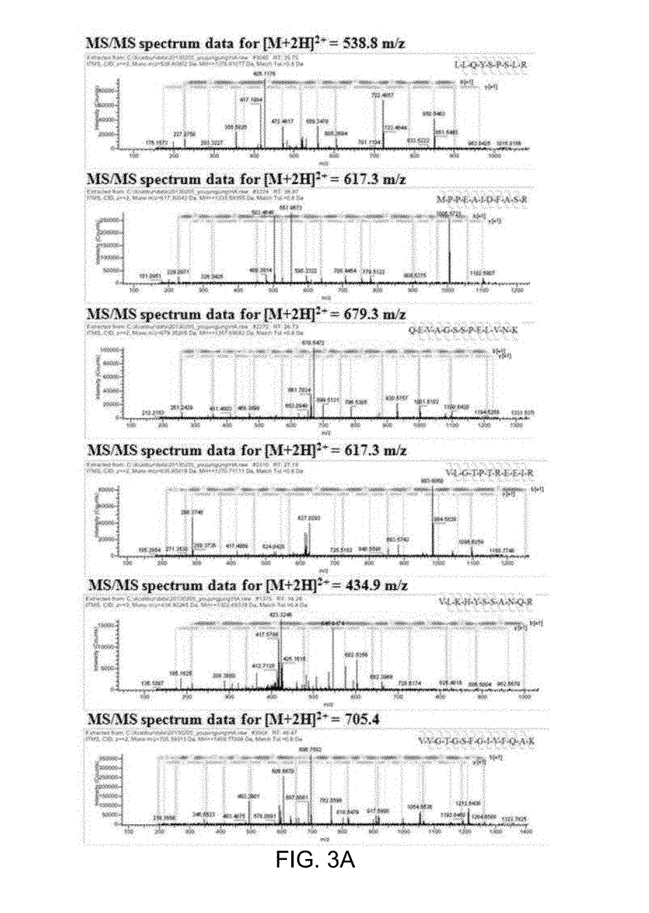

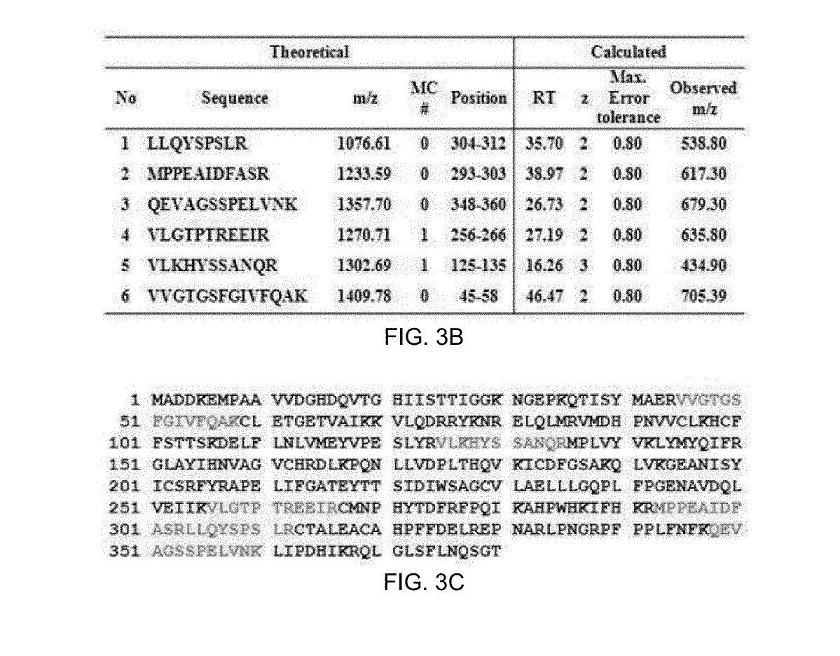

Band 1 was identified as BIN2 protein (FIG. 3). Protein specifically immunoprecipitated in BIN2-HA or bin2-6D-HA, indicating that our purification was successful. A new band of 100 kD was also recognized only in protein extracts of the BL-treated transgenic line (bin2-6D-HA and BIN2-HA) and not in extracts of wild-type seedlings (FIG. 2a).

Differential expressed bands to the bin2-6D-HA and BIN2-HA transgenic plants were identified and excised, and then subjected to MALDI-TOF/MS.

The new proteins were also identified as two E3 ligases encoded by Arabidopsis genes At5g62970 (GenBank accession number: gill 5242584; NP_201102.1) and At5g56690 (GenBank accession number: gi|15241209; NP_200480.1), which we named BR F-box 1 and 2 (BRF1 and BRF2) by MALDI-TOF/MS fingerprint analysis

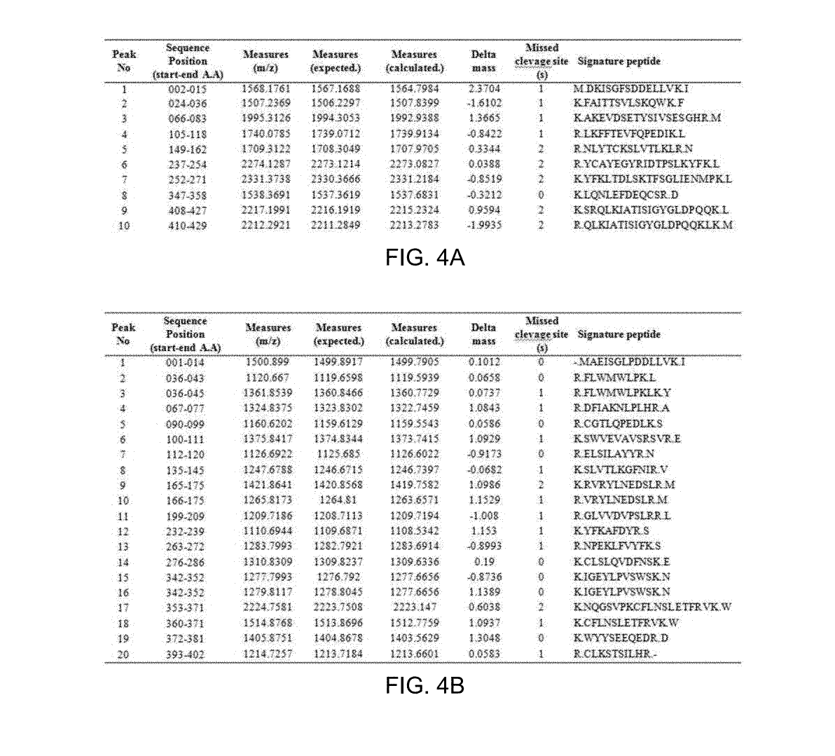

A list of signature peptides of BRF1 and BRF2 is shown in FIG. 4(SEQ ID NOs: 12-21 and 22-41). BRF1 and BRF2 in our search were also identified with a high score after searching even with the complete m/z list (FIG. 4 and FIG. 2c).

BRF1 and BRF2 shared 31%, 44% amino acid sequence identity (FIGS. 2c and 2d). In this experiment, we used a combination of IP based on the high-affinity immobilized HA-antibody and LC/MALDI-TOF/MS to identify proteins regulating the stability of BIN2.

Compared with other techniques to identify interacting proteins, the best advantage is that the IP combined with LC/MALDI-TOF/MS allows protein complex isolation under various conditions, therefore allowing functional studies in which the post-translational modification of proteins in the complex can be examined (Drewes and Bouwmeester, 2003). The IP combined LC/MALDI-TOF/MS analysis identified BRF1 and BRF2 as a BIN2-interacting protein.

To further confirm this result, we investigated the protein-protein interaction by a yeast two-hybrid assay. We confirmed that BIN2 specially interacted with the BRF1 protein on SD/-L-T-H medium supplemented with 1.5 mM 3-AT in yeast two-hybrid assays (FIG. 2e). Interaction in yeast indicates that BRF1 also regulates BIN2 through similar mechanisms.

In conclusion, our data show that BIN2 interacts with BRF1 in vitro, while a direct interaction of BRF1 could not be confirmed in vivo. Overexpression of the Arabidopsis BRF1 led to wild-type (Col-0) phenotypes distinguishable from BIN2-HA lines

3. BRF1 and BRF2 Homologs Will Play Key Roles in the BIN2 Stability

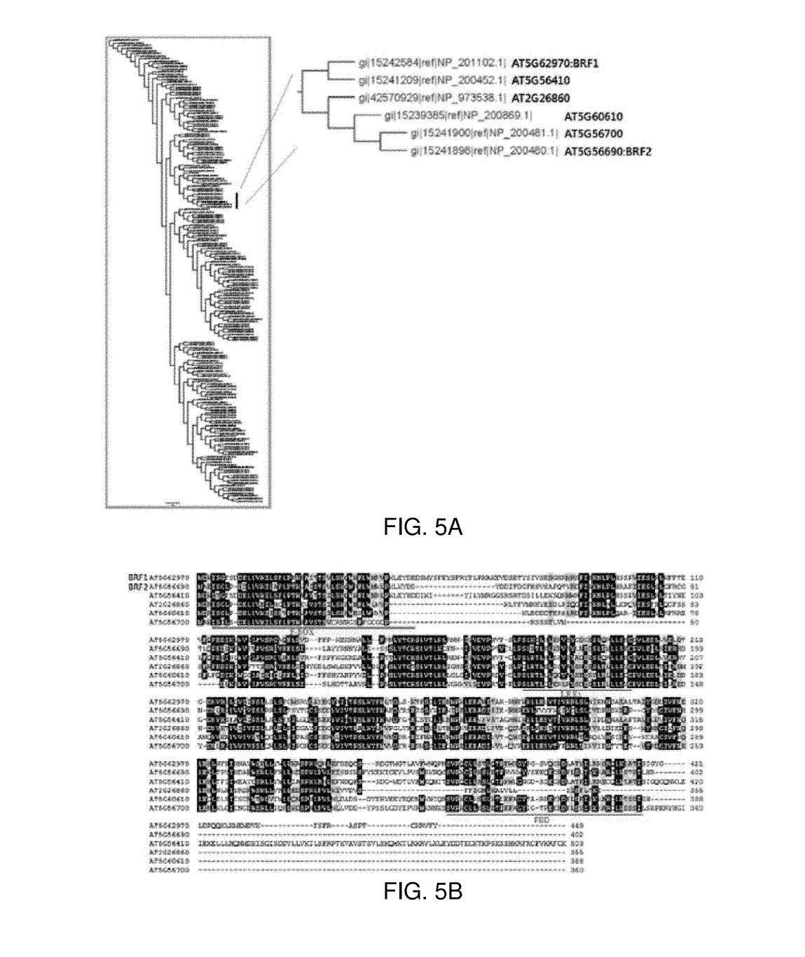

To investigate the phylogenetic relationships of BRF1 and BRF2 proteins with BRF1 and BRF2 homologene sequences, a phylogenetic tree was constructed using the neighbor joining method. Interestingly, BRF1 and BRF2 proteins (accession number; NP_201102.1 and NP_200480.1) also clustered within a phylogenetic tree as depicted in FIG. 5a, because they share such high sequence similarity with specific F-box homologous protein (FIG. 5b).

BRF1 and BRF2 encode an F-box protein consisting of F-box, leucine-rich repeats (LRRs), F-box and Leucine Rich Repeat (FBD) (FIG. 5b). These results suggest that BRF1 and BRF2 are most likely a BIN2-interacting protein. E3 ligases are key enzymes in the ubiquitination process, as they recognize different substrates for ubiquitination (Hershko and Ciechanover, 1998). F-box proteins, a component of an E3 ligase, constitute a large superfamily in plants and play important roles in controlling many biological processes.

The N-terminal F-box domain of the F-box protein is responsible for binding to Skp1, while its C-terminal protein binding domain binds targeted substrates to confer substrate specificity (Cardozo and Pagano, 2004; Ho et al., 2006; Zheng et al., 2002).

The shared F-box domains at the N terminus, F-box proteins carry leucine-rich repeats (LRRs) domain at their C terminus for substrate recognition. LRR repeats are arc-shaped β-α repeats that mediate protein-protein interaction (Enkhbayar et al., 2004; Kobe B and Kajava, 2001; Smith et al., 1999). Pairwise sequence alignment is used to identify regions of similarity that may indicate functional, structural and/or evolutionary relationships between two biological sequences (protein or nucleic acid).

Pairwise sequence alignment also revealed that the entire amino acid and nucleotide sequence of BRF2 shows the highest percent identity 49.2% and 64.2% to BRF1 (accession number: NP_201102.1 and NM_125691.1) followed by AT2G26860 (NP_973538.1: 52.9% and NM_125691.1: 66.7%), AT5G56410 (NP_200481.1: 50.5% and NM_125024.1: 64.8%), AT5G56700 (NP_200481.1: 59.8% and NM_125053.2: 73.8%), and AT5G60610 (NP_201102.1: 53.8% and NM_125691.1: 71.4%), four homologous genes of BRF1 and BRF2 F-box ligase, respectively (FIG. 5).

These results suggest that BRF1 and BRF2 homologs play redundant or overlapping roles in the BIN2 stability. The discovery of a bin2 mutant affecting the phosphorylation of some substrates could be a powerful tool for future development and use in crop manipulation. GSKs play roles in development, and these hormones are connected in responding to various environmental stresses. The discovery of BRF1 and BRF2 also provide to help of integrating multiple hormonal signals. The combination of these genetic and proteomic data also demonstrates that BRF1 is a key component of the BR signaling pathway.

Acknowledgments

This research was supported, in part, by grants from the Cooperative Research Program for Agricultural Science and Technology Development (Project No. PJ01168501), Rural Development Administration, and the National Research Foundation of Korea (NRF), grant No. 2015R1A2A1A10051668 to S.C. and 2013R1A1A2059445 to Y.J.J.

### REFERENCES

Ali A, Hoeflich K P, Woodgett J R Glycogen synthase kinase-3: properties, functions, and regulation. Chem Rev. 2001; 101:2527-2540.
Bijur G N, Jope R S. Proapoptotic stimuli induce nuclear accumulation of glycogen synthase kinase-3 beta. J Biol Chem. 2001; 276:37436-37442.
Calderbn Villalobos L I, Lee S, De Oliveira C, Ivetac A, Brandt W, Armitage L, Sheard L B, Tan X, Parry G, Mao H, Zheng N, Napier R, Kepinski S, Estelle M. A combinatorial TIR1/AFB-Aux/IAA co-receptor system for differential sensing of auxin. Nat Chem Biol. 2012; 8:477-485.
Choe S, Schmitz R J, Fujioka S, Takatsuto S, Lee M O, Yoshida S, Feldmann K A, Tax F E. Arabidopsis brassinosteroid-insensitive dwarf12 mutants are semidominant and defective in a glycogen synthase kinase 3 beta-like kinase. Plant Physiol. 2002; 130: 1506-1515.
Clough S J, Bent A F. Floral dip: a simplified method for Agrobacterium-mediated transformation of Arabidopsis thaliana. Plant J 1998; 16:735-743.
Clouse S D, Sasse J M. BRASSINOSTEROIDS: essential regulators of plant growth and development. Annu. Rev. Plant Physiol. Plant Mol Biol. 1998; 49:427-451.
Dharmasiri N, Dharmasiri S, Estelle M. The F-box protein TIR1 is an auxin receptor. Nature. 2005; 435:441-445.
Doble B W, Woodgett J R. GSK-3: tricks of the trade for a multitasking kinase. J Cell Sci. 2003; 116:1175-1186.
Dreher K, Callis J. Ubiquitin, hormones and biotic stress in plants. Ann Bot 2007, 99: 787-822.
Drewes, G, Bouwmeester, T. Global approaches to protein-protein interactions. Curr Opin Cell Biol. 2003; 15:199-205.
Enkhbayar P, Kamiya M, Osaki M, Matsumoto T, Matsushima N. Structural principles of leucine-rich repeat (LRR) proteins. Proteins 2004; 54:394-403.
Failor K L, Desyatnikov Y, Finger L A, Firestone G L. Glucocorticoid-induced degradation of GSK3 protein is triggered by Sgk and Akt signaling and controls beta-catenin dynamics and tight junction formation in mammary epithelial tumor cells. Mol Endocrinol. 2007; 21 in press.
Fu Z Q, Yan S, Saleh A, Wang W, Ruble J, Oka N, Mohan R, Spoel S H, Tada Y, Zheng N, Dong X. NPR3 and NPR4 are receptors for the immune signal salicylic acid in plants. Nature. 2012; 486: 228-232
Gampala S S, Kim T W, He J X, Tang W, Deng Z, Bai M Y, Guan S, Lalonde S, Sun Y, Gendron J M, Chen H, Shibagaki N, Ferl R J, Ehrhardt D, Chong K, Burlingame A L, Wang Z Y. An essential role for 14-3-3 proteins in brassinosteroid signal transduction in Arabidopsis. Dev Cell. 2007; 13:177-189.
He J X, Gendron J M, Yang Y, Li J, Wang Z Y. The GSK3-like kinase BIN2 phosphorylates and destabilizes BZR1, a positive regulator of the brassinosteroid signaling pathway in Arabidopsis. Proc Natl Acad Sci USA. 2002; 99:10185-10190.
He J X, Gendron J M, Sun Y, Gampala S S, Gendron N, Sun C O, Wang Z Y. BZR1 is a transcriptional repressor with dual roles in brassinosteroid homeostasis and growth responses. Science. 2005; 307:1634-1638.
Hershko A., Ciechanover A. The ubiquitin system. Annu. Rev. Biochem. 1998; 67:425-479.
Jin Y H, Kim H, Oh M, Ki H, Kim K. Regulation of Notch1/NICD and Hes1 expressions by GSK-3a/13. Mol Cells. 2009; 27:15-19.
Katsir L, Chung H S, Koo A J, Howe G A. Jasmonate signaling: a conserved mechanism of hormone sensing. Curr Opin Plant Biol. 2008; 11:428-435.
Kepinski S, Leyser O. The Arabidopsis F-box protein TIR1 is an auxin receptor. Nature. 2005; 435:446-451.
Kim T W, Guan S, Sun Y, Deng Z, Tang W, Shang J X, Sun Y, Burlingame A L, Wang Z Y. Brassinosteroid signal transduction from cell-surface receptor kinases to nuclear transcription factors. Nat Cell Biol. 2009a; 11:1254-62.
Kim W Y, Wang X, Wu Y, Doble B W, Patel S, Woodgett J R, Snider W D. GSK-3 is a master regulator of neural progenitor homeostasis. Nat Neurosci. 2009b; 12:1386-1393.
Kline K G, Barrett-Wilt G A, Sussman M R. In planta changes in protein phosphorylation induced by the plant hormone abscisic acid. Proc Natl Acad Sci USA 2010; 107:15986-15991.
Kobe B, Kajava A V. The leucine-rich repeat as a protein recognition motif. Curr Opin Struct Biol. 2001; 11:725-732.
Kockeritz L, Doble B, Patel S, Woodgett J R. Glycogen synthase kinase-3 an overview of an over-achieving protein kinase. Curr Drug Targets. 2006; 7:1377-1388.
Lee J H, Lee M J. Emerging roles of the ubiquitin-proteasome system in the steroid receptor signaling. Arch Pharm Res. 2012; 35:397-407
Li J, Nam K H. Regulation of brassinosteroid signaling by a GSK3/SHAGGY-like kinase. Science. 2002; 295:1299-1301.
Lyzenga W J, Booth J K, Stone S L The Arabidopsis RING-type E3 ligase XBAT32 mediates the proteasomal degradation of the ethylene biosynthetic enzyme, 1-aminocyclopropane-1-carboxylate synthase 7. Plant J. 2012; 71:23-34
Meares G P, Jope R S. Resolution of the nuclear localization mechanism of glycogen synthase kinase-3: functional effects in apoptosis. J Biol Chem. 2007; 282:16989-17001.
Moon J, Parry G, Estelle M. The ubiquitin-proteasome pathway and plant development. Plant Cell. 2004; 16:3181-3195.
Olsen J V, Schwartz J C, Griep-Raming J, Nielsen M L, Damoc E, Denisov E, Lange O, Remes P, Taylor D, Splendore M, Wouters E R, Senko M, Makarov A, Mann M, Horning S: A dual pressure linear ion trap-Orbitrap instrument with very high sequencing speed. Mol Cell Proteomics 2009; 8:2759-2769.
Patel S, Doble B W, MacAulay K, Sinclair E M, Drucker D J, Woodgett, J R. Tissue-specific role of glycogen synthase kinase 313 in glucose homeostasis and insulin action. Mol Cell Biol. 2008; 28:6314-6328.
Peng P, Yan Z, Zhu Y, Li J. Regulation of the Arabidopsis GSK3-like kinase BRASSINOSTEROID-INSENSITIVE 2 through proteasome-mediated protein degradation. Mol Plant. 2008; 1:338-46.
Perez-Perez J M, Ponce M R, Micol J L. The UCU1 Arabidopsis gene encodes a SHAGGY/GSK3-like kinase required for cell expansion along the proximodistal axis. Dev Biol. 2002; 242:161-173.
Rock K L, Gramm C, Rothstein L, Clark K, Stein R, Dick L, Hwang D, Goldberg A L. Inhibitors of the proteasome block the degradation of most cell proteins and the generation of peptides presented on MHC class I molecules. Cell 1994; 78:761-771.
Ruegger M, Dewey E, Gray W M, Hobbie L, Turner J, Estelle M. The TIR1 protein of Arabidopsis functions in auxin response and is related to human SKP2 and yeast grr1p. Genes Dev. 1998; 12:198-207.
Ryu H, Kim K, Cho H, Park J, Choe S, Hwang I. Nucleo cytoplasmic Shuttling of BZR1 Mediated by Phosphorylation Is Essential in Arabidopsis Brassinosteroid Signaling. Plant Cell. 2007; 19:2749-2762.
Santner A, Estelle M. Recent advances and emerging trends in plant hormone signalling. Nature. 2009; 459:1071-1078.
Schwechheimer C, Willige B C. Shedding light on gibberellic acid signalling. Curr Opin Plant Biol. 2009; 12:57-62.
Sekimata K, Kimura T, Kaneko I, Nakano T, Yoneyama K, Takeuchi Y, Yoshida S, Asami T. A specific brassinosteroid biosynthesis inhibitor, Brz2001: Evaluation of its effects on Arabidopsis, cress, tobacco, and rice. Planta. 2001; 213: 716-721.
Sheard L B, Tan X, Mao H, Withers J, Ben-Nissan G, Hinds T R, Kobayashi Y, Hsu F F, Sharon M, Browse J, He S Y, Rizo J, Howe G A, Zheng N. Jasmonate perception by inositol-phosphate-potentiated COI1-JAZ co-receptor. Nature. 2010; 468:400-405.
Smith T F, Gaitatzes C, Saxena K, Neer E J. The WD repeat: a common architecture for diverse functions. Trends Biochem Sci. 1999; 24:181-185.
Vert G, Chory J. Downstream nuclear events in brassinosteroid signalling. Nature. 2006; 441:96-100.
Vert G, Walcher C L, Chory J, Nemhauser J L. Integration of auxin and brassinosteroid pathways by Auxin Response Factor 2. Proc Nat Acad Sci. 2008; 105:9829-9834.
Vierstra R D. The ubiquitin/26S proteasome pathway, the complex last chapter in the life of many plant proteins. Trends Plant Sci. 2003; 8:135-142.
Vierstra R D. The ubiquitin-26S proteasome system at the nexus of plant biology. Nat Rev Mol Cell Biol. 2009; 10:385-397.
Wang Z Y, Nakano T, Gendron J, He J, Chen M, Vafeados D, Yang Y, Fujioka S, Yoshida S, Asami T, Chory J. Nuclear-localized BZR1 mediates brassinosteroid-induced growth and feedback suppression of brassinosteroid biosynthesis. Dev Cell. 2002; 2:505-513.
Wheeler D L, Barrett T, Benson D A, Bryant S H, Canese K, et al (NCBI Resource Coordinators) Database resources of the National Center for Biotechnology Information. Nucleic Acids Res. 2007; 35: D5-D12.
Willige B C, Ghosh S, Nill C, Zourelidou M, Dohmann E M, Maier A, Schwechheimer C. The DELLA domain of GA INSENSITIVE mediates the interaction with the GA INSENSITIVE DWARF1A gibberellin receptor of Arabidopsis. Plant Cell. 2007; 19:1209-1220.
Wu D, Pan W. GSK3: a multifaceted kinase in Wnt signaling. Trends Biochem Sci. 2010; 35:161-8.
Xie D X, Feys B F, James S, Nieto-Rostro M, Turner J G. COI1: an Arabidopsis gene required for jasmonate-regulated defense and fertility. Science. 1998; 280:1091-1094.
Yin Y, Wang Z Y, Mora-Garcia S, Li J, Yoshida S, Asami T, Chory J. BES1 accumulates in the nucleus in response to brassinosteroids to regulate gene expression and promote stem elongation. Cell. 2002; 109:181-191.
Yin Y, Vafeados D, Tao Y, Yoshida S, Asami T, Chory J. A new class of transcription factors mediates brassinosteroid-regulated gene expression in Arabidopsis. Cell. 2005; 120:249-259.
Zhao J, Peng P, Schmitz R J, Decker A D, Tax F E, Li J. Two putative BIN2 substrates are nuclear components of brassinosteroid signaling. Plant Physiol. 2002; 130:1221-1229.
Zhao P, Viner R, Teo C F, Boons G J, Horn, Wells L. Combining high-energy C-trap dissociation and electron transfer dissociation for protein O-GlcNAc modification site assignment. J Proteome Res. 2011; 10:4088-4104.
Zhang S, Cai Z, Wang X: The primary signaling outputs of brassinosteroids are regulated by abscisic acid signaling. Proc Natl Acad Sci. USA 2009; 106:4543-4548.
Zheng N, Schulman B A, Song L, Miller J J, Jeffrey P D, Wang P, Chu C, Koepp D M, Elledge S J, Pagano M, Conaway R C, Conaway J W, Harper J W, Pavletich N P. Structure of the CuI1-Rbx1-Skp1-F boxSkp2 SCF ubiquitin ligase complex. Nature. 2002; 416:703-709.

This application contains references to amino acid sequences and/or nucleic acid sequences which have been submitted herewith as the sequence listing text file. The aforementioned sequence listing is hereby incorporated by reference in its entirety pursuant to 37 C.F.R. § 1.52(e).

1-6. (canceled)

7. A method for promoting growth of a plant, comprising:
transforming a plant cell using a recombinant vector comprising a gene to control BIN2 function which is the nucleotide sequence of SEQ ID NO: 1 or 3.

8. The method for promoting growth of a plant of claim 7, wherein the growth of the plant is greater than the growth of a non-transformed plant.

9. A transformed plant which is produced by the method of claim 7.

10. The transformed plant of claim 9, of which the growth is greater than the growth of a non-transformed plant.

11. A method for promoting growth of a plant, comprising:
transforming a plant cell using a recombinant vector comprising a gene coding a protein to control BIN2 function, wherein the protein is the amino acid sequence of SEQ ID NO: 2 or 4.

12. The method for promoting growth of a plant of claim 11, wherein the growth of the plant is greater than the growth of a non-transformed plant.

13. A transformed plant which is produced by the method of claim 11.

14. The transformed plant of claim 13, of which the growth is greater than the growth of a non-transformed plant.

15. A composition for promoting growth of a plant, comprising a gene coding a protein to control BIN2 function, wherein the protein is the amino acid sequence of SEQ ID NO: 2 or 4.


SEQUENCE LISTING (51 sequences)

SEQ ID NO: 1 - DNA, 1350 bp, Arabidopsis sp.
atggacaagatcagtgggttttctgatgatgaattgctcgtgaagatcttatcgtttcttccatttaaatttgctataacaacgagtgttttgtcgaagcaatggaagtttctttggatgcgggtgccaaaacttgagtacgatgaagatagcatgtattctttcgaatattccttccgatattttttacctaaggctaaggaggttgacagcgaaacctattctatagtgtccgaaagcggccataggatgcggtcttttattgagaagaatctgccattacatagctctccggtcatagagagcttgcgtctcaagtttttcactgaggtatttcaacctgaagatatcaaattgtgggttgaaattgcagtttctcgttgtgctcaagagctaagcgttgacttttttcccaaggaaaaacacaatgcattactgccgagaaacttgtatacatgcaaatcgcttgtgacattgaaattaagaaacaacattctcgtggatgttcctcatgttttttccctcccttccttgaaaattttgcaccttgaacgtgtgacatacggagatggagaatctcttcaacggcttttatctaattgctcggttttggaagatttggttgttgaactagatacaggtgacaatgtgagaaagttagatgttatcatcccttcattgcttagtttatcctttgggatgtctagatactgcgcttatgaagggtataggatagatactccttctttgaagtatttcaaacttacagatttaagtaaaactttctccggcttgattgagaatatgccgaagctggaggaggcaaatatcactgctcgtcacaatttcaagaagcttcttgaactggtcacatctgtcaagcgtctttcattgaacatagaaaacaacgatgcagaggccctaactgctatatatggagatgatattgtctttaatgagcttgagcatctgaattttcatattcataatgcatattggtctgagttactatactggttgctcaaagcttctcctaaactacaaaacttggagttcgatgaacaatgttcacgtgatggtactatgggtacactggcggttttctggaaccaaccgaatagtgttcctcaatgtttgttgtcgactcttcagactttcgagtggtcaggttaccctggctccgtacaagggaaagatttggcgacatatatcttgagaaagtctcgccagttaaaaattgcaacaatttcgattggatatgggttggatccacaacaaaagcttaagatggagatggatgtcaaattttctttccgtgcttcacccacatgcaaacgcgtattctattga

SEQ ID NO: 2 - protein, 449 aa, Arabidopsis sp.
MDKISGFSDDELLVKILSFLPFKFAITTSVLSKQWKFLWMRVPKLEYDEDSMYSFEYSFRYFLPKAKEVDSETYSIVSESGHRMRSFIEKNLPLHSSPVIESLRLKFFTEVFQPEDIKLWVEIAVSRCAQELSVDFFPKEKHNALLPRNLYTCKSLVTLKLRNNILVDVPHVFSLPSLKILHLERVTYGDGESLQRLLSNCSVLEDLVVELDTGDNVRKLDVIIPSLLSLSFGMSRYCAYEGYRIDTPSLKYFKLTDLSKTFSGLIENMPKLEEANITARHNFKKLLELVTSVKRLSLNIENNDAEALTAIYGDDIVFNELEHLNFHIHNAYWSELLYWLLKASPKLQNLEFDEQCSRDGTMGTLAVFWNQPNSVPQCLLSTLQTFEWSGYPGSVQGKDLATYILRKSRQLKIATISIGYGLDPQQKLKMEMDVKFSFRASPTCKRVFY

SEQ ID NO: 3 - DNA, 1209 bp, Arabidopsis sp.
atggctgaaatcagcgggctgcctgatgacttgctggtgaagatactagcgtttcttccaacaaaagttgctatatcaactagcgttttgtcaaaacaatggcggtttctttggatgtggttgcctaaacttaagtacgatgattacgatgacattactgatggtttcaattccgtgtctgcattccagacatatcgggattttattgccaagaatttgccattacacagagctcccatcatagaaagtttgagcctcggatttcgttgtggaacacttcaacctgaggatttgaaatcgtgggttgaagttgcagtttctcgtagtgtgcgtgagctaagtatccttgcatattataggaataattatgcattatcgtcgtctagtttgtatacctgcaaatcgctcgtgactttgaaaggctttaacattcgtgtggatgttcctccaacagtttgtctcctgccttccttgagaactttggaacttaaacgtgtgagatacttaaacgaagattctcttcgaatgctcctatccttttgccctgttctggaatatctaagtatagaacgacacgataatgacaatttgagagggttagttgttgatgtcccgtccttgcggcgtttatctttaacttcatatactggatgttcttctgacgattatgtaatagttacaccttctttgaagtatttcaaagcttttgattacagaagtgaaatttcctcctataagattgagaaaattccagagcttgaagaggcagatatcagtattgagcgaaatcccgagaagcttttcgtatatttcaaatctatcaaatgtctttcattacaagtagacttcaacagcaaggaagagcccgggtatgattctggtattgtcttcaatcatcttgaagaactgacgccatatataaacgacgctaattggtccaagttacttttccggttgctcaatgattcacctaaactccgagtcctcgaaatcagcaactccaagtcattttataaagaaaaaattggggagtatctaccggttagctggagcaaaaatcagggatccgttcctaaatgtttcttgaatagtctagaaactttcagggttaaatggtactacagcgaggagcaagaagatagggatttcttgagtcttatcttcaaacatgctcgatgtctgaaatctacatcaatcctgcacagatga

SEQ ID NO: 4 - protein, 402 aa, Arabidopsis sp.
MAEISGLPDDLLVKILAFLPTKVAISTSVLSKQWRFLWMWLPKLKYDDYDDITDGFNSVSAFQTYRDFIAKNLPLHRAPIIESLSLGFRCGTLQPEDLKSWVEVAVSRSVRELSILAYYRNNYALSSSSLYTCKSLVTLKGFNIRVDVPPTVCLLPSLRTLELKRVRYLNEDSLRMLLSFCPVLEYLSIERHDNDNLRGLVVDVPSLRRLSLTSYTGCSSDDYVIVTPSLKYFKAFDYRSEISSYKIEKIPELEEADISIERNPEKLFVYFKSIKCLSLQVDFNSKEEPGYDSGIVFNHLEELTPYINDANWSKLLFRLLNDSPKLRVLEISNSKSFYKEKIGEYLPVSWSKNQGSVPKCFLNSLETFRVKWYYSEEQEDRDFLSLIFKHARCLKSTSILHR

SEQ ID NO: 5 - protein, 9 aa, Arabidopsis sp.
LLQYSPSLR

SEQ ID NO: 6 - protein, 11 aa, Arabidopsis sp.
MPPEAIDFASR

SEQ ID NO: 7 - protein, 13 aa, Arabidopsis sp.
QEVAGSSPELVNK

SEQ ID NO: 8 - protein, 11 aa, Arabidopsis sp.
VLGTPTREEIR

SEQ ID NO: 9 - protein, 11 aa, Arabidopsis sp.
VLKHYSSANQR

SEQ ID NO: 10 - protein, 14 aa, Arabidopsis sp.
VVGTGSFGIVFQAK

SEQ ID NO: 11 - protein, 380 aa, Arabidopsis sp.
MADDKEMPAAVVDGHDQVTGHIISTTIGGKNGEPKQTISYMAERVVGTGSFGIVFQAKCLETGETVAIKKVLQDRRYKNRELQLMRVMDHPNVVCLKHCFFSTTSKDELFLNLVMEYVPESLYRVLKHYSSANQRMPLVYVKLYMYQIFRGLAYIHNVAGVCHRDLKPQNLLVDPLTHQVKICDFGSAKQLVKGEANISYICSRFYRAPELIFGATEYTTSIDIWSAGCVLAELLLGQPLFPGENAVDQLVEIIKVLGTPTREEIRCMNPHYTDFRFPQIKAHPWHKIFHKRMPPEAIDFASRLLQYSPSLRCTALEACAHPFFDELREPNARLPNGRPFPPLFNFKQEVAGSSPELVNKLIPDHIKRQLGLSFLNQSGT

SEQ ID NO: 12 - protein, 16 aa, Arabidopsis sp.
MDKISGFSDDELLVKI

SEQ ID NO: 13 - protein, 15 aa, Arabidopsis sp.
KFAITTSVLSKQWKF

SEQ ID NO: 14 - protein, 20 aa, Arabidopsis sp.
KAKEVDSETYSIVSESGHRM

SEQ ID NO: 15 - protein, 16 aa, Arabidopsis sp.
RLKFFTEVFQPEDIKL

SEQ ID NO: 16 - protein, 16 aa, Arabidopsis sp.
RNLYTCKSLVTLKLRN

SEQ ID NO: 17 - protein, 20 aa, Arabidopsis sp.
RYCAYEGYRIDTPSLKYFKL

SEQ ID NO: 18 - protein, 22 aa, Arabidopsis sp.
KYFKLTDLSKTFSGLIENMPKL

SEQ ID NO: 19 - protein, 14 aa, Arabidopsis sp.
KLQNLEFDEQCSRD

SEQ ID NO: 20 - protein, 22 aa, Arabidopsis sp.
KSRQLKIATISIGYGLDPQQKL

SEQ ID NO: 21 - protein, 22 aa, Arabidopsis sp.
RQLKIATISIGYGLDPQQKLKM

SEQ ID NO: 22 - protein, 15 aa, Arabidopsis sp.
MAEISGLPDDLLVKI

SEQ ID NO: 23 - protein, 10 aa, Arabidopsis sp.
RFLWMWLPKL

SEQ ID NO: 24 - protein, 12 aa, Arabidopsis sp.
RFLWMWLPKLKY

SEQ ID NO: 25 - protein, 13 aa, Arabidopsis sp.
RDFIAKNLPLHRA

SEQ ID NO: 26 - protein, 12 aa, Arabidopsis sp.
RCGTLQPEDLKS

SEQ ID NO: 27 - protein, 14 aa, Arabidopsis sp.
KSWVEVAVSRSVRE

SEQ ID NO: 28 - protein, 11 aa, Arabidopsis sp.
RELSILAYYRN

SEQ ID NO: 29 - protein, 13 aa, Arabidopsis sp.
KSLVTLKGFNIRV

SEQ ID NO: 30 - protein, 13 aa, Arabidopsis sp.
KRVRYLNEDSLRM

SEQ ID NO: 31 - protein, 12 aa, Arabidopsis sp.
RVRYLNEDSLRM

SEQ ID NO: 32 - protein, 13 aa, Arabidopsis sp.
RGLVVDVPSLRRL

SEQ ID NO: 33 - protein, 10 aa, Arabidopsis sp.
KYFKAFDYRS

SEQ ID NO: 34 - protein, 12 aa, Arabidopsis sp.
RNPEKLFVYFKS

SEQ ID NO: 35 - protein, 13 aa, Arabidopsis sp.
KCLSLQVDFNSKE

SEQ ID NO: 36 - protein, 13 aa, Arabidopsis sp.
KIGEYLPVSWSKN

SEQ ID NO: 37 - protein, 13 aa, Arabidopsis sp.
KIGEYLPVSWSKN

SEQ ID NO: 38 - protein, 21 aa, Arabidopsis sp.
KNQGSVPKCFLNSLETFRVKW

SEQ ID NO: 39 - protein, 14 aa, Arabidopsis sp.
KCFLNSLETFRVKW

SEQ ID NO: 40 - protein, 12 aa, Arabidopsis sp.
KWYYSEEQEDRD

SEQ ID NO: 41 - protein, 11 aa, Arabidopsis sp.
RCLKSTSILHR

SEQ ID NO: 42 - protein, 503 aa, Arabidopsis sp.
MDKITGFSDDELLVKILSFLPTKAAVTTSILSKQWKFLWMRLPKLEYHDDIKIYILYMRGGSRSRTDSILLEKSQRMWRFIDKNLPLHSSPVIESLRLTIYNELFQPESINLWVEIAVSRCVKELSVRFSPFKGKRDALLPTTLYTCKSLVTLKLRENILVDVPHVFCLPSLKTLHLSHVTYADEESLQRLLSNCFVLEDLVVERRVGDNVRNFAVIIPSLLSLSFEILGQCSSEEYVIHTPSLKYFKARDFGECSTCLILNMPKLEEVFVSTAGHNIKKLLESVTYVKRLSLFIPDNNAEAFTALYGDVIVFNQLEHLTFIIWEAYCSKLLYWLLIASPKLRNLEFNDQFSSDGMDTLVFWEQMITSVPQCLLSSLQTFKWLGNGDSIEGKDLATFILRNSCQLKTATISIGQGQNKLEIEKELLLHQNMDKISGISDDVLLVKILSFRPTKVAVSTSVLSKQWKYLRKRVLKLEYDDTECKTKPSKSSHKRFRCFVKRFCK

SEQ ID NO: 43 - protein, 355 aa, Arabidopsis sp.
MDRISGLPDELLVEILHCLPTKEVVSTSILSKRWEFLWLWVPKLTFVMNHYESDLPIQDFITKNLRLLKPQVIESFHLQCFSSSFKPEDIKHWVVTTISRRVRELIINYCDLSWLDKPVVLLDLPNSLYTCTSLVTLKLIGHSIIVDVPRTVSLPCLKTLELDSVAYSNEESLRLLLSYCPVLEDLTIHRDMHDNVKTLVIIVPSLLRLNLPIDGAYSCDGYVIVTPALKYLKVPGLYREDFSYLLTHMPNVEEADLSVEQDVERLFESITSVKRLSLFVLLDIEDESMYHNGIYFNQLEHLNLHIYRDNWSKLLVRLLEDSPKLRVLKIVVDVSFFIGNLKALVLLIISFLTKI

SEQ ID NO: 44 - protein, 388 aa, Arabidopsis sp.
MDRISGLPDELLVKIISFVPTKVAVSTSILSKRWESLWKWVPKLECDCTEPALRDFILKNLPLQARIIESLYLRFRRESFLFQDIKLWGGIAISHCLRELRIDFFSHYANPYVILPRSLYTCKSLVTLKLLGLGIRVDVPRDVCLPSLKTLLLQCVAYSEEDPLRLLLSCCPVLEDLVIELDDANQNVKALVVIVPTLQCLSLKIPASCSDERYLIVTPSLKYFKVEDDREIFNALIENMPELEEADIYVTQHIETLLESVTSVKRLTLRQLYNSIDEYKCRAGIVFKQLEQLELSICSDNWTKLVIWLLQNSPNLRVLNLDADSDYERYEEYEQDNWKNIQRSVPKCLKSSLKTLEFAGYTARPEERDFLSFIFKKARCLKTSSISH

SEQ ID NO: 45 - protein, 360 aa, Arabidopsis sp.
MAKISDLSDELLVKILSFLPTKEAVSTSVCRNNGSFFGCGCPNSSSTLVMDLKSWVRIAVSRCVRELSISLHDTTAAVSLPSSLYTCKSLVTLKLYGKKVLLDVPRTVFLPSLKTLQLERLRYSDEDSLRLLLSYCPVLEDLSIVREDYDNLRALVVIVPSLQRLSLEIPGNCSSDGYVIVTPSLKYFKVVDYRESMSYLIEQMPELEEADIVVLQYPEKLLESVTFFKRLSIRVIFNTYTETVYRDGIVFNRLENLKLCICNGDWSKLLIQFLKDSPNLRVLNLLVDDYPSSLGDYEPVRWKNNKSSVPKCLLESLETFEFAGYIGTPEERDFLSYIFKHARCLKSSSILSRPERYHGI

SEQ ID NO: 46 - DNA, 26 bp, Artificial Sequence
  BIN2-F primer
caccatggctgatgataaggagatgc

SEQ ID NO: 47 - DNA, 20 bp, Artificial Sequence
  BIN2-R primer
agttccagattcaagaagct

SEQ ID NO: 48 - DNA, 31 bp, Artificial Sequence
  BIN2-NdeI-F primer
tcatatgatggctcatgataaggagatgcct

SEQ ID NO: 49 - DNA, 33 bp, Artificial Sequence
  BIN2- EcoRI-R primer
tggaatttcttaagttccagattgattcaagaa

SEQ ID NO: 50 - DNA, 33 bp, Artificial Sequence
  BRF1-NdeI-F primer
ctgcatatgatggacaagatcagtgggttttct

SEQ ID NO: 51 - DNA, 33 bp, Artificial Sequence
  BRF1-EcoRI-R primer
cgggaattctcaatagaatacgcgtttgcatgt